(19) United States
(12) Patent Application Publication
(10) Pub. No.: US 2003/0074049 A1
(43) Pub. Date: Apr. 17, 2003

(54) Covered stents and systems for deploying covered stents

(75) Inventors: David M. Hoganson, Philadelphia, PA (US); John E. Nash, Chester Springs, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: Kensey Nash Corporation, Exton, PA

(21) Appl. No.: 10/288,676

(22) Filed: Nov. 5, 2002

Related U.S. Application Data
(62) Division of application No. 09/645,886, filed on Aug. 25, 2000.

(51) Int. Cl. A61F 2/06

(52) U.S. Cl. 623/1.13

(57) ABSTRACT

A covered stent for use in a vessel, duct, lumen or hollow organ of a living being. The covered stent includes a stent or framework of interconnected elongated members in the form of a hollow tube having an inner surface and an outer surface. The stent may be a coiled stent, slotted tube stent, self-expanding stent, or any other intravascular stent design and may be metal or a polymer or a combination. A cover is disposed over a portion of the stent, either on the inside surface, the outside surface or intermediate those surfaces. The cover may be a polymer and may be resorbable. The cover can be attached to the stent by wrapping a sheet of polymer material around the stent, or forming a tube of polymer material and mounting it over the stent. The cover can extend over the entire stent or only a portion of the stent and may include one or more drugs or other beneficial active agents for delivery into the body of the being. Moreover, the cover may have properties to prevent permanent occlusion of a side-branch or bifurcation when placed within a branching or bifurcated vessel and may be constructed to selectively perforate or otherwise provide an opening to allow flow in a side-branch or bifurcated vessel.c

### RELATED APPLICATIONS

This application is a Divisional of our earlier filed U.S. patent application, Ser. No. 09/645,886, filed on Aug. 25, 2000, entitled Covered Stents, Systems For Deploying Covered Stents And Methods Of Deploying Covered Stents, whose disclosure is incorporated by reference herein, and which is assigned to the same assignee as the subject invention.

### BACKGROUND OF THE INVENTION

The invention generally relates to percutaneous treatment of cardiovascular disease, specifically relating to stenting. The invention more particularly concerns a stent covered with polymer capable of releasing drugs or other therapeutic agents.

Cardiovascular disease claims nearly 1 million lives each year in the United States alone. Coronary artery disease (CAD), a form of cardiovascular disease, causes nearly 500,000 deaths in the United States annually, making it the single leading cause of death of both males and females in America. CAD is characterized by the formation of fatty deposits in the blood vessel walls. As the disease progresses, narrowing of the arteries occurs and complications due to poor blood flow arise. Over the last several decades, much research has been performed to better understand the stages of CAD, the factors that contribute to its progression, and the treatment options for the debilitating and life-threatening conditions the disease causes.

Currently coronary artery disease is treated with drugs, percutaneous intervention, or surgery. Percutaneous treatment of coronary artery disease originated with angioplasty of simple lesions and has evolved into a very advanced therapy for patients with multiple and complex lesions. In the last ten years, stenting has emerged as a standard therapy for percutaneous treatment of coronary artery disease. Like the beginning of angioplasty, stenting was initially used for discrete lesions to prevent abrupt or acute closure of the lesion after angioplasty. Research demonstrated that metal stents not only prevent abrupt closure but they also reduce the rate at which new lesions are formed at the same site, a phenomenon called restenosis. Stenting is used currently used in up to 75% of the coronary artery lesions which are treated with angioplasty.

Many companies have developed and are currently distributing metal stents for the treatment of cardiovascular disease. Many inventors have described stents and their utility for the treatment of cardiovascular disease. U.S. Pat. No. 4,788,665 (Palmaz) is an example of such disclosures and is incorporated herein by reference.

Although percutaneous treatment of coronary artery disease has continued to advance, especially stenting, there are still many limitations of this treatment modality. The restenosis rate for coronary stents is still 15-40%. The restenosis appears to be independent of stent design for metal stents. Researchers have investigated coatings for stents to reduce the inflammatory response to the foreign material but the coatings have not reduced the restenosis rate. Other limitations of current metal stents stents include the potential for distal embolization caused by lesion material entering the lumen after extrusion between the stent struts and ineffective sealing of aneurysms, perforations, and dissections.

Researchers have attempted to address the problems of restenosis by releasing drugs from the stent. Conventional stents typically include a framework of plural interconnected elongated members or sections formed as an integral unit or connected to one another. The portions or sections of the framework are commonly referred to as struts. The stent struts normally only cover approximately 15% of the vessel wall and therefore is a very inefficient vehicle for releasing a significant amount of drugs or uniformly releasing drugs which would enter and effect the vessel wall. Recent advances in stent development has led to the experimentation with covered stents or stent grafts to address some of the limitations of normal stents. A covered stent is a stent which incorporates a substantially solid membrane to provide mechanical support across the stent struts. Covered stents are able to effectively seal aneurysms, perforations, and dissections. The mechanical barrier across the stent struts also prevents material from the lesion to move through the stent struts and into the lumen during placement of the stent. This often occurs during placement of a normal stent and results in distal embolization in the treated vessel. In theory, the mechanical barrier of a stent cover can reduce restenosis of the vessel. Restenosis is often caused by intimal hyperplasia, or the uncontrolled growth of the vessel wall into the lumen of the vessel. The mechanical barrier of a covered stent may prevent excessive tissue growth from occluding the vessel. Jomed International AB (Helsingborg, Sweden) has developed a stent graft which comprises a sheet of PTFE sandwiched between two metal stents. The U.S. Pat. No.5,916,264 describe this device and are incorporated herein by reference. Although the JOMED stent graft has been successful at sealing aneurysms and perforations, it is a bulky device with a significantly larger crossing profile and reduced flexibility compared to a state-of-the-art stent. Furthermore, the stent graft evokes a significant inflammatory response and has been demonstrated to be unsuitable in small diameter vessels.

Other inventors have described similar concepts to address some of the limitations of stents. U.S. Pat. Nos. 4,739,762 and 4,776,337 (Palmaz) describe a metal stent having a polymer coating on the outside of the stent. The coating is intended to be highly elastic to expand with the stent and the coating may have openings in the coating to allow fluid to contact the stented portion of the vessel.

U.S. Pat. No.5,389,106 (Tower) describes a vascular stent constructed of platinum covered with a non-permeable, non-resorbable polymer membrane. The membrane is elastic and the combined stent and membrane are designed to be deployed in a vessel with a standard balloon catheter. The non-permeable nature of the membrane is intended to be a percutaneous treatment for vessel aneurysms.

U.S. Pat. No.5,769,884 (Solovay) describes a metal stent with a non-resorbable porous cover. The cover incorporates multiple pore sizes intended to promote uniform re-endothelization while inhibiting unwanted cellular ingrowth through the cover.

U.S. Pat. No.5,843,161 (Solovay) describes a metal stent with an elastomeric sleeve surrounding the stent. The sleeve covering for the stent is intended to be an improvement in crossing profile over other covered stents which have non-elastic sleeves which are unfolded as they are deployed in a vessel. The sleeve is considered for vessels with diameters of 4-12 mm.

U.S. Pat. No.5,755,774 (Pinchuk) describes a stent-graft which is characterized by a graft material which is expanded beyond its yield point without retaining residual stress in the material. The stent graft material may be on the inside or outside of the stent. Furthermore, the graft material is a non-resorbable polymer that may be a single sheet or constructed of multiple fibers.

Polymer materials are commonly used to absorb and release drugs or other therapeutic agents. In general, non-resorbable polymers can be used as drug delivery vehicles with the porosity and thickness of the polymer determining the rate at which the drug is released. Resorbable polymers have also been used to delivery drugs. Resorbable polymers release the drugs as the polymer is resorbed. The rate at which the polymer is resorbed controls the rate the drug is released. The polymer resorption rate is related to the polymer material, molecular weight, residual stress, and other factors.

Resorbable polymers have other advantages than simply as a drug delivery vehicle. In “Arterial Regeneration Activity After Prosthetic Implantation” Arch Surg 120;315-323, March 1985, Greisler et al. reports the uniform growth of endothelial cells over resorbable polymer vascular grafts. In approximately one month, the resorbable polymer grafts are uniformly covered by a layer of endothelial-like cells over neo-intima containing smooth-musclelike myofibroblasts. Furthermore resorbable vascular grafts have demonstrated better more complete and uniform growth of endothelial cells than standard vascular graft materials as reported by Greisler et al. in “Derivation of Neointima in Vascular Grafts” Circulation 78(suppll);1-6 -1-12, 1988. Resorbable polymers have demonstrated biocompatibility and are non-thrombogenic. Additionally, since the material is completely resorbed, there is no chronic foreign body response.

The use of resorbable polymers in combination for drug delivery and as a mechanical cover has been described in the literature. U.S. Pat. Nos. 5,102,417 (Palmaz et al.) and 5,195,984 (Schatz) describe that the coating could be made of resorbable polymer, such as polyglycolides or polylactides, and the coating could contain drugs which would be released into the lumen as the polymer resorbed.

U.S. Pat. No. 5,637,113 (Tartagila et al.) describes a metal stent with a polymer film mounted to the exterior of the stent. The film is elastic with or without pores and expands in-vivo as the stent is placed. It is recognized that attaching polymer films to metal stents is an important aspect of a covered stent. The inventors disclose several methods of mounting the film to a stent including tightly wrapping the film around the stent and bonding the film to itself by heat, solvent bonding, mechanically fastening, hooking the film material into a portion of the stent or bonding the film to elastic members encircling the film. Alternatively it is considered that the film may uncoil rather than expand to conform to the diameter of the vessel upon stent expansion. Additionally, the polymer film may incorporate drugs for release into the vessel. Finally, the polymer film may incorporate a lubricious coating on the outside to promote navigation of the covered stent into the desired location within the vessel.

U.S. Pat. No.5,707,385 (Williams) describes a metal stent with a polymer film attached to the exterior of the stent. The combined film and stent structure are delivered in a vessel with a balloon catheter. The polymer film may be resorbable or non-resorbable and drugs may be incorporated into the film for release in the vessel.

U.S. Pat. No.5,833,651 (Donovan et al.) describes a stent with a polymer and fiber composition cover on the exterior of the stent. The cover is intended to deliver genetic material to the vessel wall with a virus to promote healing of the wall. The virus can transfer genetic material to cells within the vessel to produce the necessary drugs or factors to reduce the rate of restenosis.

U.S. Pat. No.5,443,496 (Schwartz) describes a metal stent with a polymer film covering the stent. The intention of the covered stent is to seal dissections or perforations and reduce the rate of restenosis. The stent cover incorporates drugs for release into the vessel by attaching microcapsules of drugs to the stent cover.

U.S. Pat. No. 5,779,732 (Amundson) describes a metal stent with a polymer film wrapped around the stent. The film is releasably attached to the stent with a suture. Upon expansion of the stent, the film is released and uncoils to contact the vessel wall. Since the cover material does not have to stretch upon delivery to the vessel wall, alternative materials can be used over stent covers which are required to be highly elastic. Drugs may be incorporated into the cover for slow release into the vessel.

U.S. Pat. No. 5,383,928 (Scott et al.) describes a stent covered with a polymer cover designed to release drugs to the arterial wall or lumen. The sheath may be resorbable or non-resorbable polymer and is considered for the delivery of anticoagulants, growth factors, anti-growth factors, restenosis inhibitors. The sheath may be used to release multiple drugs including different drugs to the vessel wall and vessel lumen. The drug release from the polymer sheath will be dependent on the diffusion rate through the polymer for a non-resorbable polymer and the thickness of the polymer for a resorbable polymer.

Other concepts for preventing the restenosis of vessels have included a mechanical barrier without the structure of a stent. U.S. Pat. No. 4,560,374 (Hammerslag) describes an elastic liner for placement in a vessel to prevent restenosis. The synthetic liner is designed to be expanded to contact the vessel wall with a balloon tipped catheter.

U.S. Pat. No. 5,749,922 (Slepian et al.) describes a process to coat a portion of vessel with a thin layer of polymer. The polymer layer is intended to reduce restenosis through performing mechanical functions similar to a stent and then resorbing into the vessel wall leaving a remodeled vessel. The polymer applied to the vessel wall may be resorbable or non-resorbable and can be administered by applying a monomer or pre-formed polymer solution to the vessel wall, or by expanding a polymer tube in the vessel until polymer tube contacts and supports vessel lumen. Heat or radiation (i.e. UV radiation) may be used to alter the polymer in-vivo to achieve conformance of the polymer to the vessel wall. Additionally drugs may be released from the polymer to further effect the healing of the vessel and prevention of restenosis. Finally, cellular material may be incorporated into the polymer material. The cellular material may be genetically modified to release drugs or other agents to further aid in the repair of the vessel wall.

One limitation of a covered stent is the potential for occluding side-branches when stenting bifurcating vessels. Placing a covered stent over a critical side-branch can cause ischemia and cell death of the tissues perfused by the side-branch. The U.S. Pat. No. 6,007,573 (Wallace et al.) describes a half layer stent which can be aligned within a vessel to block an aneurysm on one side of the vessel wall and provide an open structure on the opposite side of the vessel wall to allow flow to a branch blood vessel.

The prevention of restenosis, the sealing of aneurysms and perforations, and the prevention of distal embolization during stenting are serious needs facing percutaneous treatment of cardiovascular disease. These and other limitations of current stent technology have been addressed in theory by covered stent concepts. While the aforementioned covered stent concepts have fundamentally addressed several limitations of stenting, they suffer from one disadvantage or another. The JOMED stent has found limited clinical success but is very cumbersome to delivery and is ineffective in treating small or bifurcating vessels. Other concepts are limited by choice of materials or method of attachment of cover to stent. It is the intent of this invention to overcome these and other shortcomings of the prior art.

### SUMMARY OF THE INVENTION

In accordance with one aspect of this invention there is provided a covered stent for placement in a vessel, duct, lumen or hollow organ of a living being. The covered stent comprises a stent and a cover. The stent is an elongated tubular member having a pair of marginal edges and an intermediate portion and comprises plural elongated support portions interconnected with one another to form an open tubular framework. The open tubular framework has an inner surface and an outer surface. The cover is disposed over at least one of the surfaces of the hollow tubular framework or within the interstices between the plural elongated support portions of the framework. The marginal edges of the covered stent are more radially compliant with respect to the vessel, duct, lumen or hollow organ than the intermediate portion of the covered stent.

In accordance with another aspect of this invention there is provided a covered stent. The stent comprises a stent and a cover. The stent is also in the form of an open tubular framework having an inner surface and an outer surface. The cover being disposed over at least one of said surfaces of said hollow tubular framework or within the interstices between the plural elongated support portions of the framework. The cover has an opening in it to enable the covered stent to be used in a vessel, duct, lumen or hollow organ of the living being that has a side-branch or bifurcation to another vessel, duct, lumen or hollow organ so that flow of a fluid from the vessel, duct, lumen or hollow organ to the other vessel, duct, lumen or hollow organ is not blocked by the cover.

In accordance with another aspect of this invention there is provided a covered stent. The stent comprises a stent and a cover. The stent is also in the form of an open tubular framework having an inner surface and an outer surface. The cover being disposed over at least one of said surfaces of said hollow tubular framework or within the interstices between the plural elongated support portions of the framework. The cover has an penetratable portion arranged to be penetrated to form an opening in the cover. The opening that is formed enables the covered stent to be used in a vessel, duct, lumen or hollow organ of the living being that has a side-branch or bifurcation to another vessel, duct, lumen or hollow organ so that flow of a fluid from the vessel, duct, lumen or hollow organ to the other vessel, duct, lumen or hollow organ is not blocked by the cover.

In accordance with another aspect of this invention there is provided a covered stent for placement in a vessel, duct, lumen or hollow organ of a living being. The covered stent comprises a stent and a cover and at least two biologically active agents. The stent is an elongated tubular member having a pair of marginal edges and an intermediate portion and comprises plural elongated support portions interconnected with one another to form an open tubular framework. The open tubular framework has an inner surface and an outer surface. The cover is disposed over at least one of the surfaces of the hollow tubular framework and comprises plural layers. The cover has an outer surface, an inner surface and at least one intermediate surface. At least one of the biologically active agents is located on either or both of the exterior and interior surfaces, at least another of the at least two biologically active agents is located on the intermediate surface.

In accordance with another aspect of this invention there is provided a system for deploying a stent within a vessel, duct, lumen or hollow organ in the body of a living being. The stent comprises a hollow expandable tubular member having a pair of marginal ends and an intermediate portion. The system comprises an inflatable balloon. The balloon is a generally cylindrical member and having a distal end portion, and intermediate portion, and a proximal end portion and is arranged to support the stent on it, with the stent's end portion being located over the distal and proximal end portions of the balloon, and with the stent's intermediate portion being located over the intermediate portion of the balloon. The intermediate portion of the balloon being of greater diameter when inflated than the distal and proximal end portions of the balloon.

In accordance with another aspect of this invention there is provided the combination of an stent and inflatable balloon for deploying the stent. The stent comprises a hollow expandable tubular member having a pair of marginal ends and an intermediate portion. The balloon is generally cylindrical in shape and has a first portion and a second portion. The balloon is arranged to support the stent thereon with one portion of the stent being located over the first portion of the balloon, and with another portion of the stent being located over the second portion of the balloon. The first portion of the balloon is of greater diameter when inflated than the second portion of the balloon.

### DESCRIPTION OF THE DRAWINGS

FIG. 1 is an isometric view of one exemplary embodiment of a covered stent constructed in accordance with this invention, wherein the covering is mounted on the exterior of the stent framework;

FIG. 2a is a transverse cross sectional view taken along line 2a-2a of the embodiment shown in FIG. 1;

FIG. 2b is a transverse cross sectional view, similar to FIG. 2a, but showing an alternative embodiment for a covered stent constructed in accordance with this invention, i.e., the covering being mounted on the interior of the stent framework;

FIG. 2c is a transverse cross sectional view, similar to FIGS. 2a and 2b, but showing another alternative embodiment for a covered stent constructed in accordance with this invention, i.e., the covering being mounted on both the exterior and the interior of the stent framework;

FIG. 2d is a trasverse cross sectional view, similar to FIG. 2a, but showing another alternative embodiment for a covered stent constructed in accordance with this invention, i.e., the covering being in the form of a laminate of more than one layer of material(s);

FIG. 2e is a transverse cross sectional view, similarto FIG. 2a, but showing another alternative embodiment for a covered stent constructed in accordance with this invention, i.e., the covering being formed of a porous material(s);

FIG. 3 is an isometric view, like that of FIG. 1, but showing another exemplary embodiment of a covered stent constructed in accordance with this invention, wherein the covering is mounted on the exterior of the stent framework, with the covering of this embodiment comprising three different materials located in respective longitudinally located sections of the cover;

FIG. 4 is a longitudinal sectional view of a diseased blood vessel in which a covered stent constructed in accordance with this invention is shown being deployed;

FIG. 5 is a somewhat enlarged sectional view taken along line 5-5 of FIG. 3, with a portion of the section of the covered stent bounded by the broken circle being shown more greatly enlarged in this figure;

FIG. 6ais an isometric view, similar to FIG. 1, but showing another alternative embodiment of a covered stent constructed in accordance with this invention;

FIG. 6b is an isometric view, similar to FIG. 1, but showing still another alternative embodiment of a covered stent constructed in accordance with this invention;

FIG. 6c is an isometric view, similar to FIG. 1, but showing yet another alternative embodiment of a covered stent constructed in accordance with this invention;

FIG. 7a is an side elevational view of a covered stent constructed in accordance with this invention shown mounted on a balloon of a delivery catheter to advance the covered stent into a lumen in the body of a living being;

FIG. 7b is an side elevational view, partially in section, of a covered stent, like that of the embodiment of FIG. 6b, shown mounted on an alternative balloon of a delivery catheter to advance the covered stent into a lumen in the body of a living being;

FIG. 7c is an side elevational view, partially in section, of a covered stent like the embodiment of FIG. 6a shown mounted on the alternative balloon like that of FIG. 7b to deploy the covered stent into a lumen in the body of a living being;

FIG. 7d is a side elevational view like FIG. 7b but showing yet another alternative embodiment of a balloon for deploying the stent.

FIG. 8 is a longitudinal sectional view of an exemplary apparatus for fabricating a covered stent of this invention in accordance with one method of this invention;

FIG. 9 is an illustration of another method of this invention for fabricating a covered stent of this invention;

FIG. 10 is an illustration of still another method of this invention for fabricating a covered stent of this invention;

FIG. 11a is an illustration of still another method of this invention for fabricating a covered stent of this invention;

FIG. 11b is an illustration of still another method of this invention for fabricating a covered stent of this invention;

FIG. 12a is an illustration of another embodiment of a covered stent constructed in accordance with this invention for delivering a drug or other beneficial agent into the body of the being in whom the covered stent is deployed;

FIG. 12b is an illustration of the fabrication of another embodiment of a covered stent constructed in accordance with this invention for delivering a drug or other beneficial agent into the body of the being in whom the covered stent is deployed;

FIG. 13 is an illustration of another embodiment of a covered stent constructed in accordance with this invention for delivering a drug or cellular seeded material agent into the body of the being in whom the covered stent is deployed;

FIG. 14a is an isometric view of another alternative embodiment of a covered stent in accordance with this invention designed to preserve fluid flow to a side-branch or bifurcating vessel or lumen;

FIG. 14b is an illustration of showing the deployment of the covered stent of FIG. 14a in the common carotid artery at the bifurcation of the internal carotid artery and the external carotid artery;

FIG. 15 is an isometric view, like that of FIG. 14a, but showing another alternative embodiment of a covered stent in accordance with this invention designed to preserve fluid flow to a side-branch or bifurcating vessel or lumen;

FIG. 16a is an isometric view of another alternative embodiment of a covered stent in accordance with this invention shown located in a vessel having a side branch during the in-vivo modification of the covered stent to preserve fluid flow to the side-branch;

FIG. 16b is an isometric view, similar to FIG. 16a, but showing the covered stent after in-vivo modification thereof to preserve fluid flow to the side-branch;

FIG. 17a is a longitudinal sectional view of the distal end of a piercing device for providing an opening in a covered stent constructed in accordance with this invention to provide access to a side-branch vessel otherwise blocked by the cover of the stent;

FIG. 17b is a view similar to FIG. 17a, but showing the device in its operative state;

FIG. 17c is a view similar to FIG. 17a, but showing the device for enabling a conventional guide-wire to extend therethough into the side-branch;

FIGS. 18a-18f are respective illustrations of a process of using a covered stent constructed in accordance with this invention to stent a lesion at the bifurcation of the left anterior descending (LAD) artery and the first diagonal branch (D1) off of the LAD including use of the piercing device of FIG. 17 to provide access to the LAD downstream of the bifurcation; and

FIG. 19 is a longitudinal sectional view similar to FIG. 4, but a showing deployment system and methodology for selectively opening the cover of a covered stent constructed in accordance with this invention to provide access to a side-branch temporarily blocked by the cover of the stent.

### DETAILED DESCRIPTION OF PREFERRED EMBODIMENTS

Referring now to the drawing wherein like reference characters refer to like parts, there is shown in FIG. 1 a covered stent 10 constructed in accordance with this invention. The covered stent 10 includes a stent body or framework 20 and a cover 22. The stent body 20 is made up of plural interconnected elongated portions or sections 24, either formed as an integral unit or assembled and connected to one another to form a hollow tubular framework having an exterior surface and an interior surface. Any conventional stent design or construction can be utilized as the stent body or framework of the covered stent 10. As is known such design/constructions can entail coil stents, slotted tube stents, self expanding stents, etc. The materials making up the stent framework can include steel (e.g. stainless steel), titanium, platinum, nitinol, MRI visible materials, plastic (resorbable or non-resorbable) or any other suitable material which can provide the necessary mechanical requirements of a stent.

Stents are normally delivered into the lumen to be treated in a collapsed state with a diameter or crossing profile which is smaller than the diameter of the lumen. The stents are then expanded with an expanding member, e.g. balloon, or are released from a constrained configuration and self-expand by nature of their construction. The covered stent 10 is also arranged to be expanded or to be self-expanding (although some non-expandable stents may be constructed in accordance with this invention for specialized applications not requiring expansion within the vessel, duct, lumen or organ). To that end, the cover 22 is preferably an elastic material which can be expanded as the stent is expanded within the vessel, duct, lumen or hollow organ (those biological structures being hereinafter collectively referred to as lumens hereinafter) of the living being. For a self-expanding stent, the stent cover can be expanded with the expansion force inherent in the stent, or the covered self-expanding stent can be expanded with a balloon as is described by Richter et al. in European Patent Application No. 97116. Once a covered self expanding stent is erected in a lumen, the stent will retain its self-expanding properties. If the cover is constructed of a resorbable material, once the material is weakened so its tensile strength is less than the radial force exerted by the self expanding stent, the stent will exert radial force on the vessel as designed. It may be useful to use a self-expanding covered stent in certain lumens, e.g. the carotids, where it is necessary to prevent permanent collapse of the stent by external mechanical force.

In addition to the elastic nature of the stent cover 22, the cover material is such that after expansion it has low residual stress to prevent the material from tearing or prematurely degrading due to residual stress. Control of the elasticity of the cover material can control the necessary inflation pressure of the covered stent. This can result in a covered stent with a very low inflation pressure which will minimize vessel trauma during stent deployment. Low residual stress in the material can be achieved with either a very elastic material (>1000% elongation) or a material which is stretched beyond its elastic limit and plastically deforms.

Cover thickness is minimized to reduce crossing profile and necessary expansion pressure of the covered stent. Generally, the cover will be thinner than 0.005 inch (0.127 mm) and more specifically thinner than 0.002 inch (0.051 mm). The thickness of the cover can be consistent over the length of the stent or it can vary. For example, portions of the cover 22 can be thinner adjacent the ends 20a and 20b (FIG. 1) of the stent 20 and thicker in the middle portion of the stent. A thinner cover at the ends of the stent further reduces the leading crossing profile of the covered stent when it is introduced into the lumen to be treated. Additionally a thinner cover at the ends of the stent would reduce the stiffness in that area which would decrease the compliance mismatch between the covered stent and the vessel, and thereby may reduce end-effects stenoses at the vessel-stent interface. Finally, the thickness of the cover may vary along the length of the stent to control the inflation characteristics of the covered stent. It may be advantageous for the covered stent to inflate at the ends first or conversely it may be advantageous for the stent to inflate in the middle first. Varying the thickness of the cover would vary the necessary inflation pressure for the stent in each portion and thereby control the inflation characteristics.

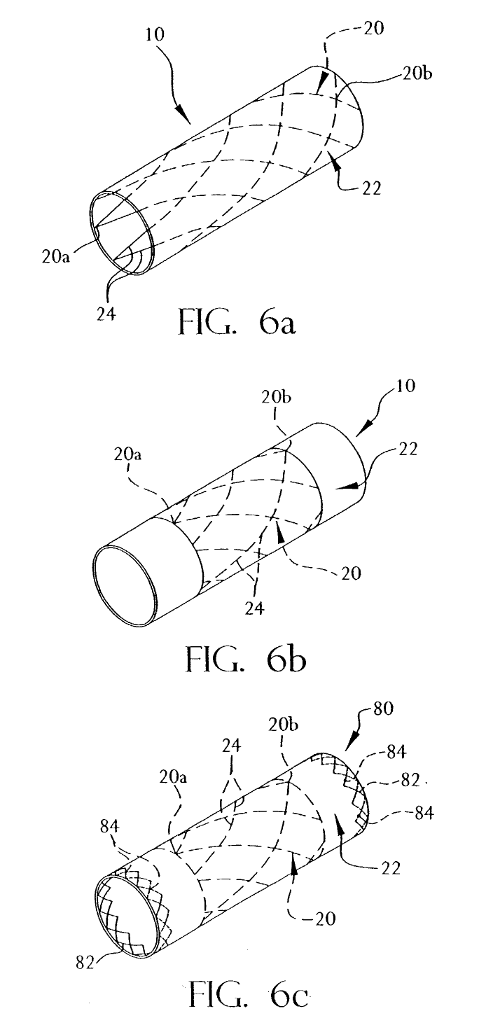

In the embodiment of the covered stent 10 shown in FIG. 1 the ends 20a and 20b of the stent 20 extend beyond the marginal edges of the cover 22. This arrangement is one of various arrangements of the position of the cover with respect to the stent. Thus, as will be seen later with respect to the embodiment of the covered stent shown in FIG. 6a, the cover of the stent can be coincident with the ends of the stent.

The material of the cover 22 may be any biocompatible material which has sufficient elasticity and is mechanically stable in-vivo. Non-resorbable polymers may provide such necessary mechanical characteristics. Examples of non-resorbable polymers are shown in the following Table 1:

TABLE 1
Non-resorbable Polymer Examples

Polyurethane
Polytetrafluoroethylene (PTFE)
Expanded Polytetrafluoroethylene (ePTFE)
Polyethylene Teraphthalate (Dacron)
Polypropylene

Resorbable polymers may also be used, as they provide many distinct advantages for this application. Resorbable polymers can be made to have very wide ranges of elasticity up to 3400% elongation at break. Furthermore they can be made to plastically deform upon elongation to minimize the residual stress in the material after expansion. The resorption rates of resorbable polymers can be controlled by varying the polymer material, molecular weight, additives, processing, and sterilization. Resorption rates can be adjusted to be shorter for applications of covered stents that require mechanical strength for only a short period of time or longer for applications that require mechanical strength to be present until a replacement vascular wall has formed. As resorbable polymers degrade, they are replaced by endothelial and smooth muscle-like cells which eventually form new vascular wall. Because they are eventually replaced by a mechanically viable vascular wall, resorbable polymers can be used as cover materials for covered stents used in the treatment of aneurysms or perforations. For example, a resorbable polymer covered stent 10 constructed in accordance with this invention may be an ideal for a percutaneously deployed stent graft for the repair of abdominal or thoracic aortic aneurysms. Examples of resorbable polymers that can be used as a cover material are shown in following Table 2. These materials are only representative of the materials and combinations of materials which can be used as stent cover material.

TABLE 2
Resorbable Polymer Examples

Polyglycolide (PGA)
Copolymers of glycolide
Polylactide (PLA)
Poly-L-lactide (PLLA)
Poly-DL-lactide (PDLLA)
L-lactide/DL-lactide copolymers
Glycolide/L-lactide copolymers (PGA/PLLA)
Poly-e-Caprolactone (PCL)
Trimethylene carbonate (TMC)
Lactide/caprolactone copolymers (PLA/PCL)
Glycolide/trimethylene carbonate copolymers (PGA/TMC)
Polyhydroxyalkonates (pha polymers)
Lactide/tetramethylglycolide copolymers
Lactide/s-valerolactone copolymers
Polydepsipeptides
PLA/polyethylene oxide copolymers
Poly-b-hydroxybutyrate (PBA)
PHBA/g-hydroxyvalerate copolymers (PHBA/HVA)
Poly-b-hydroxypropionate (PHPA)
Poly-p-dioxanone (PDO)
Poly-s-valerolactone
Methyl methacrylate-N-vinyl pyrrolidone copolymers
Polyesteramides
Polyesters of oxalic acid
Polydihydropyrans
Polyalkyl-2-cyanoacrylates
Polyvinyl alcohol (PVA)
Hydrogel
Polypeptides
Poly-b-malic acid (PMLA)
Poly-b-alkanoic acids
Polyanhydrides
Polyorthoesters
Polyphosphazenes
Tyrosine based polymers

Resorbable or non-resorbable stent cover polymer materials can undergo various processing steps to achieve the desired material characteristics. For example, the raw polymer material may initially be combined with additives which will be described later in detail. The polymer can then be formed into the desired shape through various processes. Melt processes which can be used include extrusion, compression molding, and injection molding. The polymers can also be formed using solvent casting or other processes using solvents, such as freeze drying. These processing steps can result in porous or non-porous polymer materials in the form of sheets, films, tubes, fibers or other desired constructions. The materials then can undergo heat or chemical treating to further optimize their properties.

Through compounding or other methods of mixing, the cover material can be a combination of multiple resorbable materials, multiple non-resorbable materials, or a combination of resorbable and non-resorbable materials. The materials can be a homogenous mixture of polymers or the cover could be composed of resorbable polymer in one portion and non-resorbable polymer in another.

In addition to pure polymer materials, additives may be combined with the polymers to improve their mechanical, biological, or resorption characteristics. One example of additives would be plasticizers which can alter the mechanical performance of polymers to make them more elastic or deform more plastically. U.S. Pat. No. 5,525,646 (Lundgren et al.) discloses a resorbable polymer combined with a plasticizer which modifies the mechanical performance of the polymer. Another additive may be nanoparticles which increase the strength and may change the resorption properties of polymers. Additives can be incorporated into the polymers with standard melt compounding, solvent mixing, or other processes. Examples of plasticizers and nanoparticles are shown in following Tables 3 and 4.

TABLE 3
Polymer Plasticizers

Citrate esters
adipate esters
epoxidized soy oil
acetylated coconut oil (EPZ)
linseed oil
Dioctyl adipate
acetyltri-n-butyl citrate
actyltriethyl citrate
acetyltri-n-hexyl citrate
n-butyryltri-n-hexyl citrate
stearic acid
calcium stearate
Bis-2-methoxyethyl phthalate
acetoxytriethyl citrate
Glyceryl triacetate
ethyl benzoate
diethyl phthalate
dibutylphthalate
bis-2-methoxyethyl phthalate
Polyurethane
Glycolide
Lactide
Camphor
benzoic acid-2-hydroxyacetate
hexamethylbenzene
1,2-cyclohexadione
Ethyl-, butyl-, and hexyl-esters of acetylated citric acid
ethyl-terminated oligomers of lactic acid

TABLE 4
Nanoparticles

Silica
Clay
Metals
Metal Oxides

Beyond polymer materials, other biocompatible and resorbable materials can be used as the cover material 22. For example, collagen, elastin, fibrin, and thrombin are materials which are present in the human body in various forms, and which could also be used as the cover material for a stent constructed in accordance with this invention. Other biomaterials not derived from a human body may be used as a material for the stent cover. An example of such material would be chitosan.

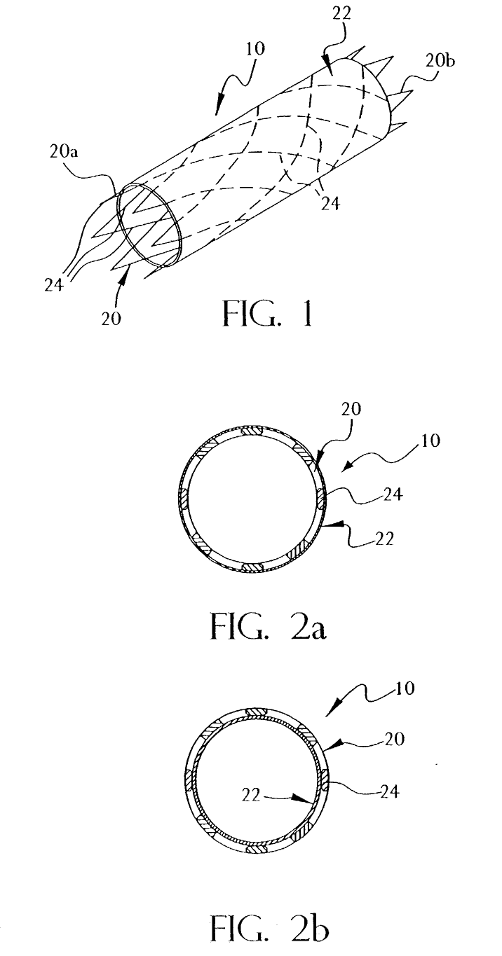

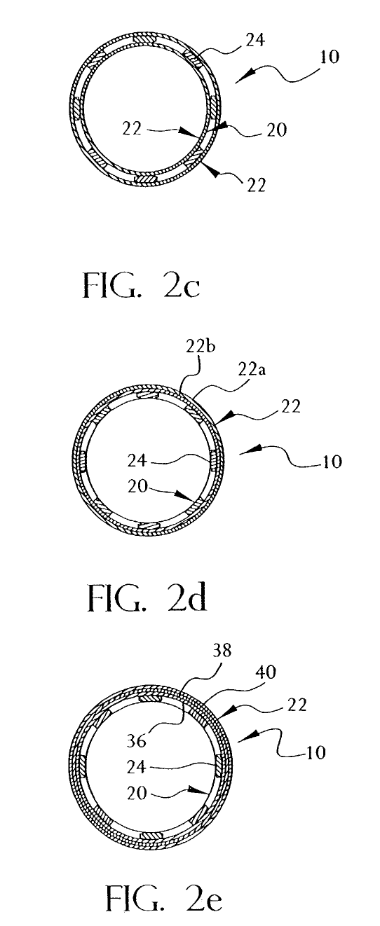

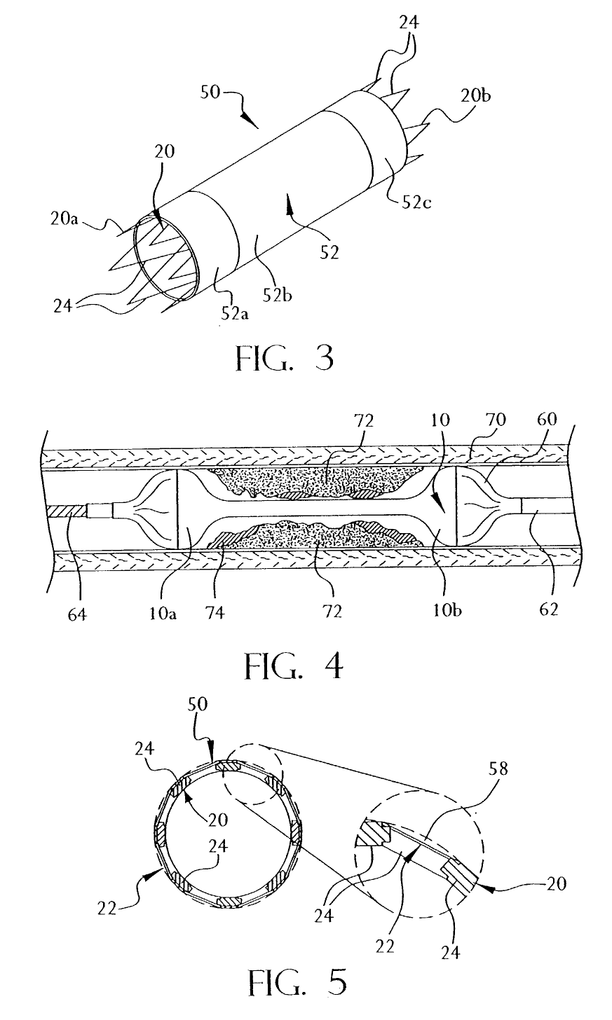

In FIG. 2a there is shown a simplified cross-sectional view of the covered stent 10 embodiment of FIG. 1. In this embodiment the cover 22 is located on the outside of the stent 20. This is just one exemplary arrangement. Thus, the cover 22 may be located on the interior of the stent 20, as shown in FIG. 2b. So too, a cover 22 may be located on the exterior of the stent framework and a cover 22 on the interior of the stent framework, such as shown in FIG. 2c. Moreover, the cover 22 may be located in the interstitial spaces between the portions or sections 24 of the framework, so as effectively being in the “middle” of the stent. The stent cover can be in the form of a single layer or a laminate of materials. In FIG. 2d there is shown an embodiment of a covered stent 10 whose cover 22 is in the form of a laminate located on the exterior of the framework. In that embodiment the cover comprises a laminate of two layers 22a and 22b. It should be pointed out at this juncture that such an arrangement is merely exemplary of the various laminated constructions contemplated by this invention. Thus, the cover can include as many layers or plies of any material, as desired. Moreover, the laminated cover can be located on the stent in any location that a single layer cover can used. The construction and details of manufacturing a laminated cover will be described later. Suffice it to say that the cover can be formed of one or more layers of material with one or more materials comprising each layer.

The material making up the cover 22 is preferably non-porous to prevent the release of inflammatory agents and embolic debris into the affected lumen. However, this characteristic is not mandatory. Thus, the material can also be porous with a controlled porosity. A porous cover can be constructed of any of the above mentioned materials. The porosity of the cover can be controlled to encourage the rapid growth of cellular material on the cover. Moreover, the porosity of the cover can vary along the length or thickness of the cover. The cover 22 can comprise a combination of porous and non-porous layers or regions. For example, as shown in FIG. 2e the innermost layer 36 of an exteriorly-located cover 22 (i.e., a cover located on the outer surface of the framework 20) can be porous with a defined porosity. The next or intermediate layer 38 of the cover can be a non-porous barrier layer, and the outer-most layer 40 of the cover, i.e, the portion of the cover 22 adjacent the vessel wall, can be porous with a defined porosity which can be the same or different than the porosity of the inner layer 36.

One advantage of a covered stent over a non-covered stent is the ability of the covered stent to uniformly seal a lesion or damaged area against the vessel wall. This may include loose portions of the lesion, e.g. thrombus, intimal flaps, cholesterol clefts, or portions of the lesion which may otherwise have been forced through the struts of a normal stent on balloon expansion. These particles or portions of the lesion which are forced or extruded through the stent struts or framework during balloon expansion can be sheared off during balloon deflation or balloon removal and may progress down stream in the vessel and cause distal embolization. Many distal protection devices have been developed to capture these distal embolization particles that are released during stenting or most commonly post dilatation of stents. The available distal protection systems utilize a balloon to block distal flow during stenting and then remove the debris before restoring flow or a distal filter which captures the debris released during stenting. Distal embolization has long been understood to be a major component of complications in carotid and saphenous vein graft stenting and is just now being recognized as a significant component of complications in coronary stenting as well. A covered stent may reduce or even prevent the release of particles which might cause distal embolization. A covered stent which effectively prevents the release of debris may even obviate the need for distal protection devices in most interventional procedures.

In the embodiments shown in FIGS. 1 and 2 the cover 22 is shown as being in the form of a continuous cylindrical wall. Depending upon the material making up the cover, the thickness thereof, and the manner of application of the cover to the stent body or framework 20, portions of the cover 22 between adjacent struts or sections 24 of the framework may be somewhat planar in shape, such as shown in FIG. 5. These planar areas of the cover may produce respective cavities between them and the inner surface of the vessel or lumen wall to further deter the release of debris from the lesion being stented, as will be described later.

As described previously, this invention can control the inflation characteristics of the stent system which can be used to minimize the potential for distal embolization. Inflating the covered stent 10 at the ends 10a and 10b first can be controlled by the balloon catheter (e.g. controlling balloon folding, balloon shape, wall thickness, retaining means), the stent (design or thickness of the struts in a particular portion), or the cover 22. Thickness of the material for the cover can vary as described previously, and the material can also be different in different portions of the cover as shown in FIG. 3. In particular in FIG. 3 there is shown a covered stent 50 constructed similarly to stent 10, i.e., having a stent body or framework 20 but having a different cover 52. In this embodiment the cover 52 comprises three different materials 52a, 52b, and 52c mounted on the exterior surface of the stent 20 in respective longitudinally located sections of the cover. One or more materials can be used along the length of the cover to vary the mechanical or other properties of the cover. A cover material which is thinner or more elastic at the ends of the stent can allow the ends of the stent to expand first and provide a mechanical seal to prevent extrusion of the lesion out of the ends of the stent when the middle of the stent expands. Thus, the sections 52a and 52c can be made thinner than section 52b of the cover.

In FIG. 4, a covered stent constructed in accordance with this invention is shown being deployed within the lumen of a lesion 72 in a blood vessel 70. This illustration shows the benefits of the covered stent having the ability to be expanded at its ends 10a and 10b by a deployment balloon to trap the lesion 72 and associated debris 74 against the vessel wall. In particular, a covered stent 10 is mounted on a balloon catheter 62 which is advanced into the target vessel 70 over a guidewire 64. The balloon 60 initially expands at the ends which results in expansion of the ends 10a and 10b of the covered stent 10 against the vessel wall 70. This traps the lesion material 72 and any loose debris 74 in the middle of the covered stent and prevents release of this material into the lumen. Although a covered stent which is approximately or equal to the length of the lesion may be used, it may be advantageous to use a covered stent which is substantially longer than the lesion to ensure that the ends of the stent are adjacent to a portion of the vessel which has less or no disease. This will improve the seal of the covered stent against the vessel with less inflation pressure and may minimize the extrusion and axial movement of the lesion material.

An additional feature which can prevent distal embolization is shown in FIG. 5. The cover of the stent shown in FIG. 5 includes the heretofore mentioned generally planar portions between adjacent struts 24 of the stent's framework 20. These planar portions can produce respective cavities 58 between the cover 22 and the inner surface of the lesion 72 when the covered stent 10 is expanded. In particular, these cavities 58 are created as the cover material 22 stretches in the portions between the stent struts 24 but does not slide over the struts on expansion of the covered stent. The size and number of the cavities may vary according to the design of the stent. The cavities serve to trap the lesion material and prevent it from moving along the axis of the stent and extruding out of the ends of the stent during expansion. The cavities may be aligned in an alternating pattern such that movement of the lesion material from the middle of the stent towards the ends is minimized (not shown). According to the stent design, the cavities may be smaller or absent at the ends of the stent to provide a mechanical seal at the ends of the covered stent to prevent the release of material (not shown).

Other variations of the covered stent 10 are possible to achieve characteristics which will result in improved clinical outcomes. For example, as shown in FIG. 6a, the cover 22 can extend to the ends 20a and 20b of the stent 20 and be flush with the end struts 24 making up the strut's framework. This can improve the distribution of force at the ends of the stent and may result in lower restenosis due to end effects.

An additional method of reducing the compliance match of a stent structure is to reduce the thickness and/or width of the stent struts at the ends of the stent. Thinner stent struts at the ends of the stent would reduce radial compliance of the stent at the ends and therefore reduce the compliance mismatch between the stent and the vessel. This improvement over stents may be combined with a stent cover which results in a covered stent with improve compliance match at the ends of the covered stent. Alternatively, in addition to having variable strut thickness and/or width, the ends of the struts could be constructed of a different material all together. The material used at the ends of the stent would result in reduced compliance at the ends of the stent with the positive associated effects discussed previously.

FIG. 6b shows another embodiment of the covered stent 10. In this embodiment the cover 22 extends beyond the ends 20a and 20b of the stent 20. This construction may result in an improved compliance match between the ends of the covered stent and the vessel wall which may result in reduced restenosis due to end effects. The material used for the ends of the cover may vary in thickness as previously discussed so the compliance of the material decreases between the end of the cover 22 and the beginning of the framework of the stent 20. This results in a smooth transition in compliance between the vessel wall and the end of the stent. If the cover is constructed of a resorbable material, the resorption rate of the material can be made such that the compliance transition effect would be in place long enough to prevent end-effects stenoses at the ends and then the material of the cover would degrade.

One potential limitation of extending the cover beyond the end of the stent is that the radial support of the stent is not present to ensure sealing of the cover against the wall of the vessel. This may result in blood flowing between the cover and the vessel wall and the blood pressure may force the end of the cover into the lumen of the vessel. This potential complication can be prevented while still achieving a smooth compliance transition between the vessel wall and the end of the supporting stent. To that end FIG. 6c shows a further embodiment of a covered stent 80 constructed in accordance with this invention for ensuring that the cover seals against the wall of the vessel at the ends of the cover. The stent is similar in construction to the stent shown in FIG. 6b, except for the inclusion of a pair of additional supporting members 82 at the respective overhanging ends of the cover 22. The supporting members can be of any suitable construction. In the embodiment shown they comprise respective stents 82 and are mounted within the interior of the cover at the ends of the cover and spaced from the ends 20a and 2b of the central stent 20. The end stents 82 provide an additional supporting structure for the cover 22 to ensure complete contact between the ends of the cover 22 and the vessel wall. These supporting end stents 82 can be constructed in the same manner as the central stent 20, but can be of different material or thinner material as to have higher compliance than it stent. In this case, the cover 22 is used to locate and support the end stents 82 during placement and erection. A unique design of the end stents 82 can be used to maximize compliance and minimize radial recoil while achieving sufficient radial strength to ensure sealing of the cover material to the vessel wall. Additionally, one or both of the end stents 82 may incorporate a radiopaque marker 84 to identify the ends of the covered stent 80 since the end stents may be too thin to be sufficiently radiopaque. While the embodiment 80 shown includes two supporting end members 82, such is not mandatory. Thus, a supporting member 82 may be on one or both ends of the stent. In this regard, it may only be necessary for a supporting member or end stent to be on the proximal end of the stent as long as the distal portion of cover which extends beyond the end of the stent is optimally expanded.

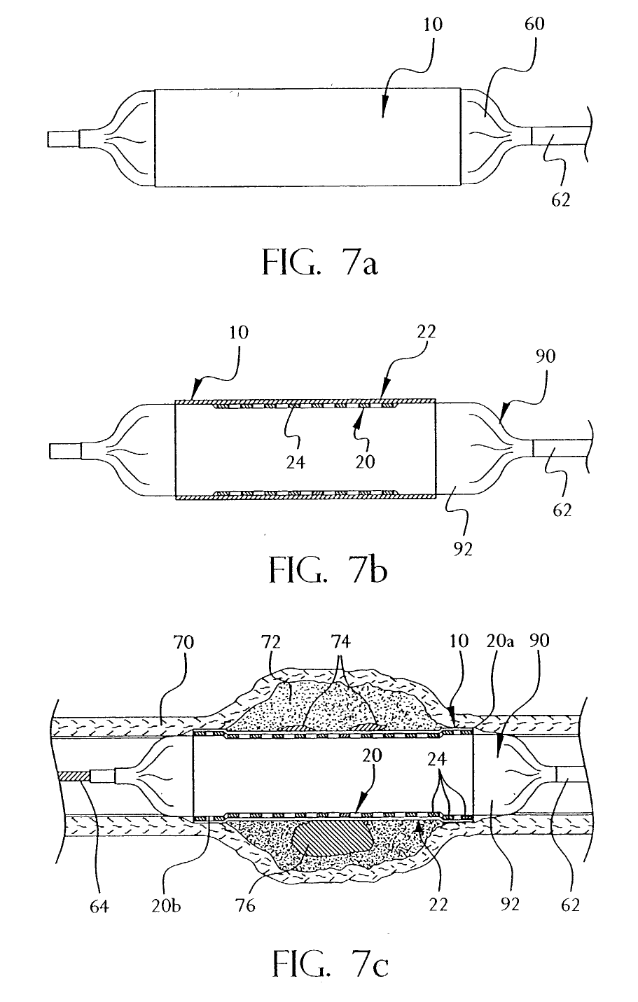

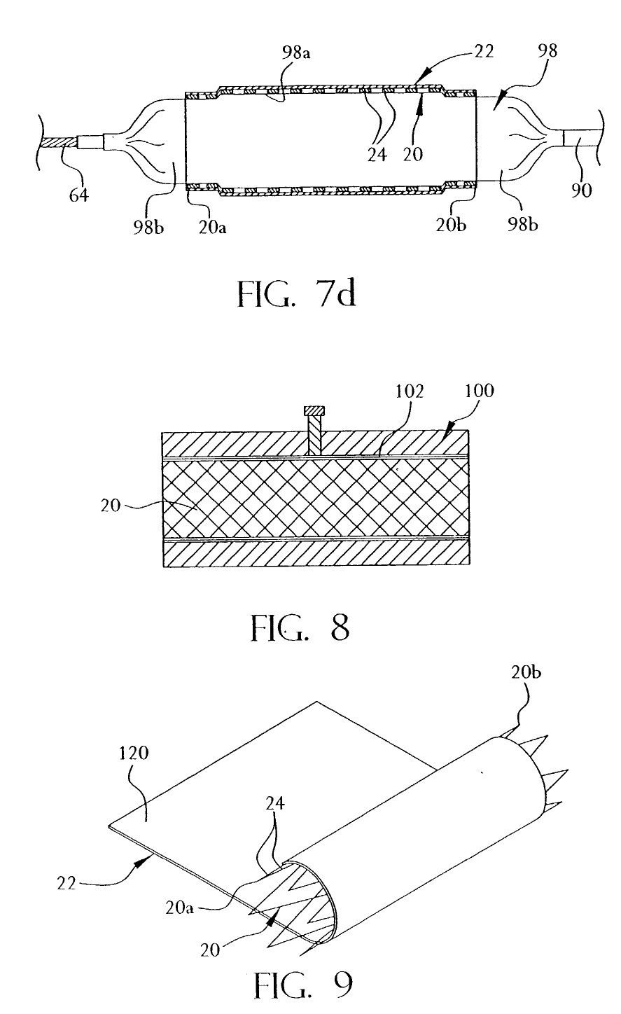

As previously described, deployment of the covered stent can be accomplished by inflating the stent with a balloon. This is shown in FIG. 7a. In particular, a covered stent constructed in accordance with any embodiment of this invention is mounted on a standard cylindrical balloon 60 of a delivery catheter 62 and advanced into a lumen to be stented. The balloon 60 is inflated and the covered stent 10 expands to contact the lumen wall.

In FIG. 7b, there is shown another catheter delivery system 90 which includes alternative balloon 92 to optimally deliver a covered stent 10 like that of FIG. 6b where the cover 22 extends beyond the end of the stent 20. A covered stent 10 like that of FIG. 6b, if attempted to be deployed, e.g., inflated, with a standard cylindrical balloon 60 like that of FIG. 7a, the ends 94 of the cover 22 extending beyond the ends 20a and 20b of the stent 20 may not be expanded to the same diameter as the portion of the cover which is on the stent 20. Therefore the ends 94 of the cover 22 may not fully contact the vessel wall. This may result in blood flowing between the vessel wall and the cover and forcing the ends of the cover into the lumen as previously discussed. The balloon 92 shown in FIG. 7b compensates for the unsupported cover portions 94 by expanding them more than it expands the stent 20. This results is full contact of the cover only portion 94 with the vessel wall. To accomplished the increased expansion of the end portions 94 of the cover, the ends of the balloon 92 are larger in diameter than the central portion thereof to adequately expand the overhanging cover material 94 while expanding the central stent 20.

The covered stent may also be used to prevent a plaque or other lesion material from becoming unstable or to prevent an unstable plaque from rupturing or releasing material into the lumen which may cause ischemia or a myocardial infarction (heart attack) or stroke. Currently, most vascular occlusions are visualized only with a fluoroscope during a diagnostic or interventional procedure. Fluoroscopy can only identify regions of stenoses and cannot assess plaque structures in the vessel wall. Sometimes intravascular ultrasound (IVUS) is utilized to obtain an image of the vessel wall. This mode of imaging can, to a limited degree, identify plaques which have large lipid or thrombus necrotic cores covered by a fibrous cap. These plaques with cores may be present in lesions that are only significantly (>50%) on non-significantly (<50%) occluding the lumen of the vessel. Regardless of the degree of occlusion at time of imaging, these necrotic cores present in plaques are at risk of becoming unstable, opening, and releasing the contents of their cores into the lumen of the vessel causing distal embolization of the released material. New imaging technology is under development to better identify these plaque structures which may become unstable and result in distal embolization. MRI catheters with imaging coils are under development which show a cross-sectional image of an artery and can precisely identifies necrotic cores. A covered stent could be placed over these plaque structures with necrotic cores to prevent rupture of the cores. The combination of identification of these necrotic cores and the treatment of effective sealing of these cores could greatly reduce the risk of heart attack or stroke for someone with significant cardiovascular disease. This treatment strategy could result in significantly better health for persons with cardiovascular disease and a significant cost savings over current treatment strategies.

FIG. 7c shows the delivery system 90 of FIG. 7b deploying a covered stent 10 like that shown in FIG. 6a. That covered stent has its supporting framework 20 extend the full length of the cover 22. Thus, expansion of the larger diameter ends of the balloon will tend to effectively seal the ends of the covered stent 10 to the vessel wall 70 and more effectively seal stenotic material 72 against the vessel wall. Expanding a covered stent into a lesion with soft, loose, or friable material 74 may result in axial movement of the lesion material 72, 74 and extrusion of the material out of the ends of the covered stent 10 as previously discussed. Expanding a covered stent 10, into a lesion 72 with a necrotic core 76, may result in rupture of the necrotic core 76 and release of the thrombus, lipid material or other contents. Since this material is likely to be in a liquid or loose gel form, it is more likely to extrude out of the ends of the covered stent unless additional measures are taken to ensure effectively sealing of the ends of the covered stent. Since the ends of the balloon 92, are larger in diameter than the center of the balloon, the ends of the covered stent 10 should be very securely sealed to the vessel wall 70. Use of a stent framework 20 with little or no elastic recoil is very important to retaining the sealing benefits achieved by this balloon delivery system 90. Furthermore, it is important that the delivery system inflates at the ends first as previously discussed and shown in FIG. 4. An additional feature of this balloon construction is that it can incorporate different wall thicknesses along the length of the balloon to vary the compliance of the balloon in different areas. For example, if the system is being used to inflate a covered stent into a lesion which contains a necrotic core and is very fibrous or possibly calcified, a high inflation pressure may be necessary to adequately dilate the lesion. This may result in the lesion material being forced axially along the covered stent with significant force. The ends of the covered stents may then need to have additional sealing force to ensure prevention of release of any material. Therefore it may be advantageous for the ends of the balloon where the diameter is larger to have slightly higher compliance and be slightly more responsive to a change in diameter with increasing pressure than the middle portion of the balloon. Making the balloon slightly thinner at the ends where the diameter is larger would increase its compliance and thereby increase the sealing pressure of the ends of the stent during high pressure dilation of a difficult lesion.

Alternative to the balloon design 92 shown in FIG. 7c, a balloon 98 with a middle portion 98a is larger than the ends 98b of the balloon as shown in FIG. 7d, may be advantageous. A covered stent 10 is mounted on the balloon 98 at the distal end of the delivery catheter 90. When the balloon 98 is expanded within the vessel, the middle portion 98a of the balloon expands to a slightly larger diameter than the ends 98b of the balloon. Often when a stenosis is dilated during stenting, especially during direct stenting, high pressures are necessary to fully dilate the lesion and position the stent uniformly against the vessel wall. Since balloon materials are not completely non-compliant, high-pressure expansion into a lesion which is situated in the middle portion of the balloon results in additional expansion of the balloon at all portions, hence the ends of the stent are expanded larger than the nominal diameter of the vessel. Once the lesion dilates, the stent expands to the slightly larger diameter of the balloon at high pressure. This results in over-stretching of the ends of the stent into a portion of the vessel which may not be diseased. The end result is a stent of very different compliance than the vessel wall which has been over-dilated into the vessel at the ends (and middle). The resulting end-effects restenosis should not be surprising. The balloon with a larger diameter in the middle portion may reduce the over expansion of the stent at the ends and may result in successful high-pressure dilation of the vessel without over expansion of the ends of the stent into the vessel wall.

One potential limitation of deploying a covered stent using fluoroscopic guidance is the inability to determine the exact contour of the vessel and the regions of the vessel which contain plaque or other deposits and the regions of the vessel which are free of disease. Cardiovascular disease can be very pervasive and often vessels can have continuous plaque deposits along the length of the vessel and only one area which has a significant stenosis. The regular use of IVUS to identify plaque, necrotic cores, and non-diseased vessels will greatly increase the effectiveness of a covered stent. Certainly the covered stent would best be placed with any necrotic cores near the middle of the stent and with sufficient additional length at the ends of the stent to capture a normal or necrotic core lesion without loss of material into the lumen. Delivery of a covered stent under MRI or other advanced guidance with precise knowledge of the vascular structures would be ideal. The covered stent may incorporate electronic resonating circuits such as is being developed by Simag GmbH (Berlin, Germany) or other technologies to improve visualization of stents with MRI.

As previously discussed, various processes can be utilized to manufacture polymers in to a desired form. These polymer embodiments can then be mounted to a stent or constructed with a stent to form a covered stent. Many different polymer embodiments can be used to form a covered stent. Polymer can be formed in a tubular shape and then mounted to an existing stent to form a covered stent. One method for achieving this is to manufacture a polymer tube with residual stress incorporated in the tube which very closely matches the diameter of the stent. The polymer tube can then be placed over the stent and heated. When heated, the polymer tube will radially shrink and compress against the stent. A covered stent is then formed which has sufficient adherence of the cover material to the stent. Accordingly, the tube could be mounted to the stent by a combination of uniform radial compression and heat. Another method of securing a polymer tube to a stent structure is described by Banas et al. in Patent Cooperation Treaty (PCT) international application number PCT/US95/10752. This covered stent embodiment utilizes the elastic recoil properties of an ePTFE graft to secure such graft to a stent utilizing only the recoil properties and inherent friction without the need for adhesives or sutures to retain the graft of the stent.

Another method of constructing a stent cover from a tubular construct is by freeze drying a polymer solution. To that end a polymer can be easily dissolved in a solvent creating a polymer solution of varying viscosity depending on the solvent and amount of polymer present in the solution. The polymer solution can then be freeze dried to produce a porous polymer construction. This method can be used to form a porous polymer cover directly on a metal stent 20.

In FIG. 8 shows an unexpanded metal stent 20 located in a mold 100 with a cavity of controlled size 102. The mold is filled with a polymer solution and freeze dried. The result is a porous polymer cover 22 incorporated into the stent wall. As the stent is expanded, the cover can expand and act as a mechanical barrier as previously discussed. Alternatively, a non-porous cover can be formed on a stent in a similar manner. Rather than freeze drying the polymer solution and stent in the mold, the mold can be placed in a vacuum chamber and the solvent removed from the film. The result is a covered stent with the cover on the inside, middle, and outside of the stent body 20 or any combination as determined by the mold design. Finally using this manufacturing concept, a cover consisting of a combination of porous and non-porous portions could be constructed. A non-porous polymer cover can be placed on the stent prior to putting the stent in the mold. The cover can be attached by various methods discussed in this application. A polymer solution can be added to the mold where the polymer material of the cover could be stable in the solvent of the polymer solution added to the mold. The mold could be freeze dried and the result would be a cover with porous and non-porous portions. The porous portions of the cover may be inside or outside the non-porous portion or a combination thereof. The advantages of such a construction have been previously discussed.

The polymer material for a covered stent can also be constructed as a thin film. This film can be formed by extrusion, compression molding, injection molding, or solvent casting. The films generally can be thinner than 0.005 inch (0.127 mm), more specifically thinner than 0.002 inch (0.051 mm), and most preferably thinner than 0.001 inch (0.0254 mm). The thin films can be mounted to a stent to form a covered stent in a variety of ways.

For example, as shown in FIG. 9, a single piece of film 120, the width of the stent (or more or less as previously discussed) can be wrapped tightly around the stent 20 one or more times to form the cover. The film 120 can then be adhered to itself, to the stent 20, or both through a number of bonding methods including but not limited to heating the polymer above its T_{g} (glass transition temperature), a combination of heat and compression, heating the polymer while wrapping it in tension around the stent, heating or melting the polymer with laser, infrared or ultrasonic energy, or solvent bonding the polymer to itself and/or the stent. Other adhesives such as fibrin, polymer, or cyanoacrylate glue can be used to bond the polymer to itself or the stent. As previously discussed, one or more layers of polymer or other material can be used to form the stent cover. These materials may utilize one or more bonding methods to secure the cover material to itself or to the stent. If the cover material is adequately secured to itself and is in tightly applied to the stent, bonding directly to the stent may not be necessary.

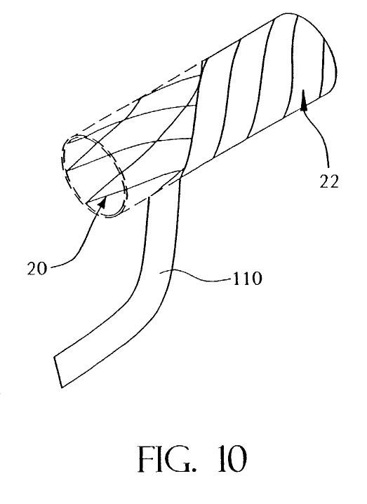

An additional method of covering a stent with a film material is shown in FIG. 10. This method uses a film 110 for the covering having a width less than the length of the stent 20 where the film 110 is helically wrapped around the stent 20. With this method, the film 110 can overlap itself or lie adjacent to itself on the stent framework. The film 110 can then be bonded to itself or the stent by one of the previously described methods.

Additionally, a film can be wrapped around the stent and secured with strips or bands along the free ends or around the circumference of the stent as is shown in FIGS. 11a and 11b. A similar concept is described in U.S. Pat. No.5,700,286 (Tartagila et al.) except the preferred embodiment of that patent included a relatively inelastic polymeric film secured by elastic bands at the free end of a wrapped film or around the ends of the wrapped film. The elastic bands would stretch to allow the polymeric material surrounding the stent to uncoil.

In FIG. 11a, there is shown a covered stent 200 having a cover 22 in the form of a polymer sheet wrapped about the stent framework 20. A strip of material 202 is provided to secure the free end 204 of the wrapped polymer cover 22 to itself. Since the polymer film making up the cover 22 is elastic, it is preferable that the strip 202 have similar or less elasticity than the polymer material. The strip can be bonded to the material using heat, polymer glue, solvent bonding, or other techniques described above.

In FIG. 11b, the there is shown a covered stent 300, like that of FIG. 11a, but which includes a polymer sheet wrapped about the stent framework and held in place by plural bands 302. The bands 302 extend around the wrapped sheet adjacent the ends of the stent 20 and are preferably of similar material as the polymer sheet with similar or less elasticity. Rather than the bands stretching as the stent is expanded, the bands 302 can have regions 304 incorporated in them where the band is designed to break or pull apart on deployment, e.g., expansion, of the stent. The band 304 is secured to the polymer sheet to form the cover with one of the previously described bonding methods so it does not come off of the covered stent 10 and embolize the vessel. Furthermore, the bands 302 are thin so they do not significantly increase the cross-sectional area of the covered stent 300.

In addition to its mechanical advantages, a covered stent constructed in accordance with any embodiment of this invention can also be used to locally delivery drugs to the vessel wall and/or lumen. The drugs can be incorporated into the cover materials(s), applied as a coating to the cover material and/or stent, incorporated into microspheres or small particles, or any combination thereof.

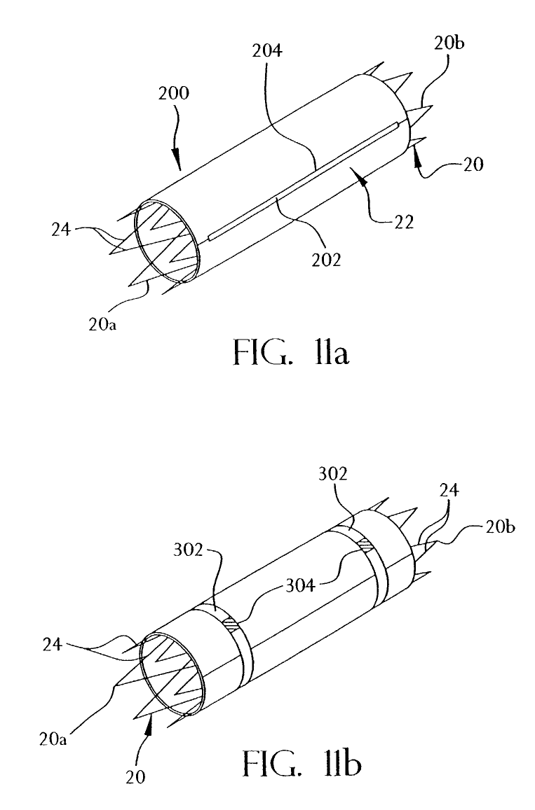

In FIGS. 12a and 12b, there are shown covered stents with drugs incorporated in them. In particular, in FIG. 12a there is shown a simplified illustration of a covered stent 10 constructed in accordance with any of the embodiments of this invention and having drugs particles 116 incorporated into the material making up the cover 22. One or more drugs can be incorporated into the cover material, and/or a coating on the cover material or stent. A combination of drugs and incorporation or application to the covered stent can result in the delivery of independent drugs to the vessel lumen and the vessel wall.

In FIG. 12b, there is shown a laminate or wrapped construction of covered stent 10 like that constructed in accordance with the method illustrated in FIG. 9, and with drugs or other beneficial agents 116 incorporated on the inside of the film 120 forming the cover 22. The agents may be located between the layers of the film forming the cover and/or on the outside of that film to be on the outside of the cover when the fabrication of the covered stent is complete.

Additionally, the drugs or agents incorporated into various portions of the cover (inside the cover, between the layers, outside the cover, etc.) may vary according to location. Different drugs or combinations of drugs/agents could be used at each location to achieve optimum therapeutic or other benefit. For example, an antigrowth factor could be applied to the outside of the cover to reduce cellular proliferation, VEGF could be applied below the outer surface of the cover but within the cover to encourage endothelial cell growth and a IIb/IIIa inhibitor could be placed on the inside surface of the cover to prevent platelet accumulation and thrombosis.

An alternative method of incorporating drugs/agents into the cover may entail first incorporation of drugs/agents into carrier particles, such as microspheres, and then incorporation of the microspheres into the cover in a matter describe above. The carrier particles may be made of the same or different material than the cover and may release the drugs/agents at uniform or variable rates relative to each other.

Additionally, the drugs/agents may vary in type, concentration, or method of incorporation along the axis of the covered stent. For example, a certain agent may be in higher concentration at the ends of the stent than the middle portion of the stent to prevent end-effect-restenosis.

It should be pointed out at this juncture that the embodiments of FIGS. 12a and 12b are only examples of the various covered stents constructed in accordance with this invention that can be utilized to deliver a drug or other beneficial agent into the body of the being in whom the stent is deployed.

Examples of drugs or other beneficial agents which may be delivered by the covered stents of this invention are listed in the following Table 5.

TABLE 5
Drug Examples

Anti-platelet agents
IIb/IIIa inhibitors
Ticlid
Coumadin
Aspirin
Heparin
Low-molecular weight heparin
Thrombin inhibitor
Anti-thrombogenic agents
Anti-inflammatory agents
Antibiotics
Growth factors
Fibroblast Growth Factors (FGF)
Vascular Endothelial Growth Factor (VEGF)
Platelet Derived Growth Factor (PDGF)
Endothelial Cell Growth Factor (ECGF)
pr39
MAC-1
Anti-growth factors
Growth factor antagonists
Antisense
Anti-restenosis drugs
Anti-proliferation drugs
Heparin sulfate proteoglycan
Beta blockers
Calcium channel blockers
Nitric oxide (NO)
Nitroglycerin
Vasodilator
Angiotensin Converting Enzyme Inhibitors (ACE inhibitors)
Anti-virus drugs
Genetic material
Adnovirus with genetic material

In addition to drugs, coatings may be added to the covered stent to increase lubricity of the outside of the covered stent for smooth delivery of the covered stent into the vessel. The coating may increase lubricity, decrease thromoboginicity, decrease platelet deposition, or provide other advantages to the covered stent. The coating may also be used as a mechanical barrier to protect underlying cellular material which may be incorporated onto the cover material as will be described in detail later. Examples of possible coating materials are listed in Table 6.

TABLE 6
Coating Material Examples

Hyaluronic acid
Silicon oil
Polyethylene Oxide
Polyethylene glycol
Polyethylene acetate
Polyvinyl pyrrolidone
Polyvinyl alcohol
Polyacrylamide
Polyanhydrides
Hydrophylic polymer

Consistent with the embodiments shown in FIGS. 12a and 12b, or other embodiments of this invention, cellular material may be incorporated into the covered stent. Cellular material may be delivered in combination with or independent of drug delivery. The cellular material may be present on the inside of the cover, outside of the cover, or incorporated within the cover in a porous construct, laminate or other such embodiment. The cellular material may be added to the covered stent immediately prior to implantation or may be grown on the covered stent in the days or weeks prior to implantation so more mature cells are in place when the cover is implanted. If the cells are seeded on the covered stent several days or weeks prior to implantation, the covered stent with only the cover or the complete construct may be placed in an in-vitro setup where blood or a blood substitute medium is circulated through the covered stent at increasing pressure to acclimate the cells to the intravascular environment. The cell-seeded cover may be in this in-vitro setup at physiologic pressure and flow for several days prior to mounting to a delivery system and implantation within the body. Cell seeding techniques have been developed for a variety of cell types. For example smooth cell seeding of biodegradable grafts is described by Yue et al. in “Smooth muscle cell seeding in biodegradable grafts in rats: A new method to enhance the process of arterial wall regeneration” in Surgery 103:206-212, February 1988. Examples of cellular material that may be seeded on covered stent are listed in the following Table 7.

TABLE 7
Cellular Material Examples

Endothelial Cells
Smooth muscle cells
Fibroblasts
Epithelial cells
Skeletal muscle cells
Genetically altered cells
Progenitor cells
Stem cells
Blood cells
Cells with altered receptors or binding sites

Alternatively, the cells could be initially grown on a sheet of polymer or other substrate and then wrapped around a stent prior to implantation. Layers of different cells could be wrapped around, inside or incorporated within a stent. For example endothelial cells, and smooth muscle cells, collagen and elastin could be isolated (from an animal, donor patient, or actual patient to be treated) and individually grown on a substrate material. Once the cells are mature, the layers of cells could be wrapped around, inside, or incorporated within a stent. One embodiment may include a layer(s) of endothelial cells on the inside of the stent, a layer(s) of smooth muscles outside the stent and a thin layer of collagen and elastin on the outside of the smooth muscle cells. The combined embodiment may be further prepared by placing it in the in-vitro setup with escalating pressure and flow previously described to condition it to intravascular conditions. The cells may be genetically altered to produce excessive amounts of certain proteins, enzymes, or other factors.

As previously noted, cover materials with drug or cellular material incorporated may be combined with cover materials without drug or cellular material to make a suitable cover for a covered stent of this invention. It is possible that the delivery of the covered stent may damage some seeded cellular constructs and therefore it may be necessary to include non-seeded cover materials on the inside or outside or within layers of cellular materials to protect and/or support the cellular seeded materials.

Furthermore, drug loaded or cellular seeded materials may not retain the desired mechanical characteristics of a covered stent (e.g. elasticity). Drug loaded or cellular seeded materials may need to be combined with other cover materials to ensure that the cover has the necessary mechanical characteristics as described previously.

In FIG. 13 there is shown by a simplified illustration an embodiment of a covered stent 400 including the combination of drug loaded or cellular seeded material 402 and non-seeded material 404 to form a complete stent cover 22. The drug loaded or cellular seeded material 402 may be applied to the non-seeded material 404 in patches, strips, or another discontinuous manner to minimize the deformation of the drug loaded or cellular material 402 upon expansion of the non-seeded material 404.

One potential limitation of using a covered stent in a blood vessel is the presence of bifurcations or side branches which a covered stent may isolate from the treated vessel. This blocking of side-branch vessels may lead to ischemia and potentially cell death for tissues without adequate collateral supply. For example, a covered stent placed in the left main coronary artery and extending into the left anterior descending coronary artery would occlude the circumflex coronary artery and cause ischemia and potentially a heart attack and death. However, a covered stent provides many advantages over non-covered stenting in bifurcating vessels as previously described, therefore it is necessary to develop methods and devices to adapt covered stents so they are applicable to bifurcating vessels.

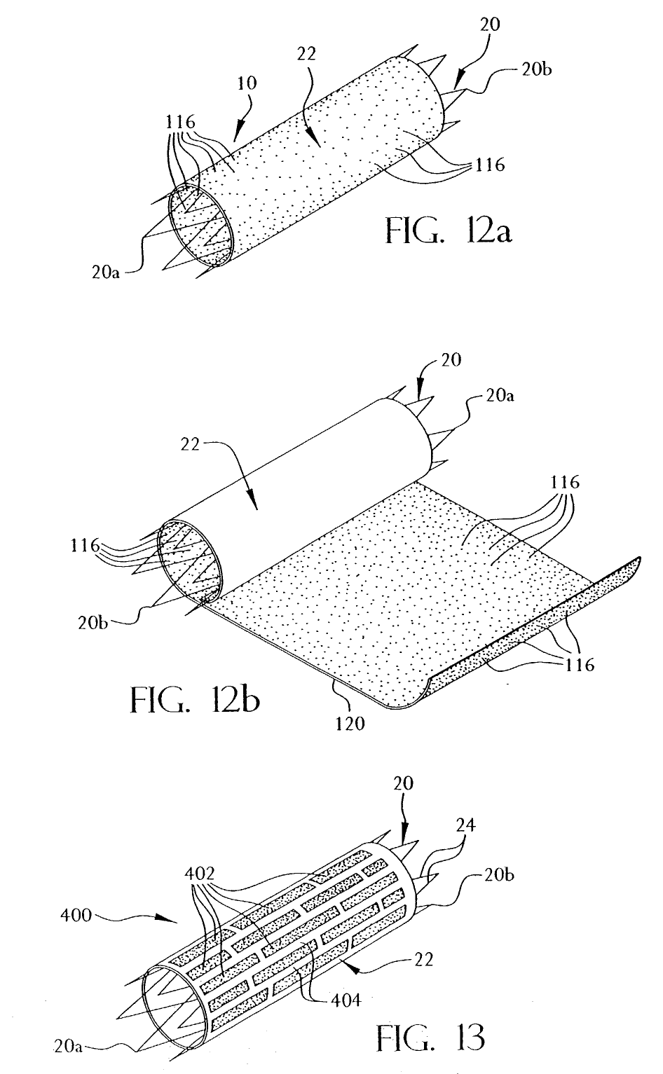

FIG. 14a shows one exemplary embodiment of a covered stent 500 designed to preserve fluid flow communication to a side-branch or bifurcating vessel. To that end the covered stent includes one or more open areas or windows. In the exemplary embodiment 500 the stent has a pair of cover sections 502 mounted on the exterior of the stent's framework contiguous with the respective ends 20a and 20b of the stent, but spaced apart from each other to form a window 504 therebetween. Each of the cover sections 502 can be in the form of any of the covers previously described and can be fabricated by any of the methods previously described. In the embodiment shown the window extends around the entire periphery of the covered stent 500. This is merely exemplary. Thus, the window may only make up a portion of the periphery of the covered stent. Moreover, the window needn't be located in the center of the covered stent, but can be at any desired location. Further still, plural windows can be utilized, if desired. In any case the window(s) provide open areas in the covered stent where blood can access and perfuse a side-branch or bifurcated vessel.

The covered stent 500 (hereinafter referred to as a “windowed stent”) may, if desired, include very noticeable radiopaque markers 506 to identify the bounds of the window(s) portions. Radiopaque markers could also be utilized on the delivery system catheter or balloon (not shown) to further identify the window(s) of the covered stent 500. The length of covered and uncovered (i.e., window) portions of the stent and the diameter of the stent can vary, depending on the target vessel or other lumen.

In FIG. 14b, the utility of the windowed stent 500 is shown deployed in a carotid artery. In this regard, the windowed stent 500 is shown placed in the common carotid artery 508 and extends into the internal carotid artery 510 while passing over the bifurcation 512 to the external carotid artery 514. The window portion 504 is located over the bifurcation takeoff of the external carotid artery 514. This preserves the external carotid artery and simultaneously provides the advantages of a covered stent to the common and internal carotid arteries. A similar covered stent construction can be used in coronary, cerebral or other peripheral arteries. As mentioned above a covered stent with multiple openings or windows to accommodate multiple side-branches can also be constructed in accordance with this invention. Once the covered stent 500 is in place and the side-branch is perfused, the portion of the stent covering the side-branch may be modified to optimize the flow in the side-branch. In particular, a guidewire (not shown) may be inserted into the side-branch through the stent struts 24 and a balloon catheter may be placed in the side-branch across the stent. The balloon can be inflated to move the stent struts and any partially occluding cover material away from the opening of the side-branch. This action would also provide clean access to the side-branch for later access or intervention. Furthermore, if there is significant bifurcation disease, which is common, a second covered stent with an open middle portion can be deployed across the bifurcation into the second vessel (in the above case, the external carotid artery) to treat the disease in the proximal portion of that vessel while providing the advantages of a covered stent. Balloon modification of the open portion near or at the bifurcation may again ensure optimum flow in both vessels. The stent framework in the window portion 506 of the stent may have a different construction than the covered portion of the stent to optimize passage of a guidewire through the expanded stent struts and balloon dilatation of the expanded struts to optimize flow into the side-branch.

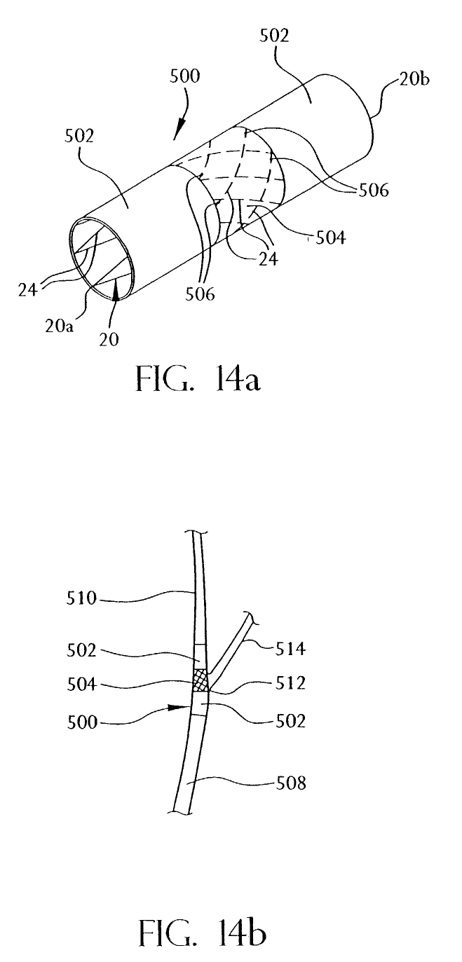

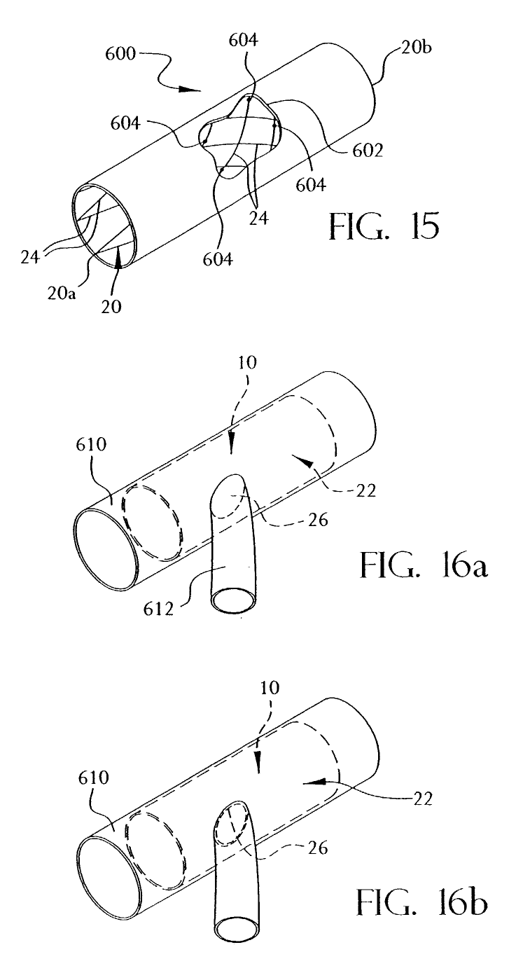

FIG. 15 shows another side-branch saving (windowed) covered stent embodiment 600. The covered stent 600 is similar to the covered stent 500, except that its cover material is not in the form of two section separated from each other to form a central window. In particular, the cover of the covered stent 600 is a single cover 22 like that described earlier having an opening or window 602. The window is confined to a predetermined portion of the periphery of the cover, i.e., it does not extend about the entire circumference. The opening or window 602 thereby exposes the open framework of the stent 20 located thereunder to allow perfusion of a side-branch vessel. The covered stent 600, like the covered stent 500, can include a system of radiopaque markers 604 to align the opening 602 with a side-branch. When the covered stent 600 is inflated during its deployment, the opening 602 is aligned with the side-branch and flow in the side-branch is preserved. As described for the embodiment in FIG. 14b, post delivery balloon modification of the bifurcation may be necessary to ensure optimum flow in both vessels.

Another embodiment of the side-branch saving covered stent is shown in FIGS. 16a and 16b. In FIG. 16a, there is shown a covered stent 10 constructed in accordance with this invention or any other non-windowed stent of this invention (i.e., a stent having a non-windowed cover) placed in a target vessel 610 which contains a side-branch 612. When the covered stent 10 is deployed, flow in the side-branch is blocked by the cover 22 since the material of the cover is substantially solid. In order to provide a fluid (blood) flow path to the side-branch 612, the covered stent 10 can be modified in-vivo (as will be described hereinafter) to open a portion 26 of its cover leading to the side-branch as shown in FIG. 16b to thereby allow perfusion of the side-branch.

Opening the cover 22 of the covered stent 10 at the location of the side-branch can be accomplished in a number of ways. For example, when the covered stent is placed over the side-branch 612, the pressure differential between the target vessel 610 and the side-branch is equivalent to the blood pressure. The material making up the cover 22 can be chosen so this pressure is enough to cause the cover to locally tear in the area of the occluded side-branch and allow blood to flow into the side-branch. This would be a selective mechanism of controlled cover tearing since the vessel wall would support the cover at all portions except in the area of the blocked side-branch. This mechanism of side-branch perfusion would result in the perfusion of all major side-branches momentarily blocked by the covered stent. This embodiment may be advantageous over previously disclosed side-branch saving techniques as the entire wall opposite the side-branch and even the ostium of the side-branch are treated by the covered stent material. As described for previously discussed side-branch saving mechanisms, post delivery balloon modification of the bifurcation may be necessary to ensure optimum flow in the side-branch vessel. The material for the covered stent previously described which selectively tears at body temperature and with a pressure differential at or below blood pressure may not be suitable for all situations in which a covered stent might be deployed. If a covered stent was deployed over a perforation or aneurysm, the aforementioned material may be ineffective in treating the defect. Additional methods and embodiments for establishing flow in temporarily blocked side-branches which overcome this limitation will be discussed later.

Another method for accessing and perfusing a side-branch which is temporarily blocked by a non-windowed covered stent constructed in accordance with this invention is to utilize a piercing device to provide an opening in the cover at the location of the side-branch to provide access to the side-branch. One such device 170 is shown in FIG. 17a. The piercing device 170 basically comprises an outer tubular structure 172 and an inner tubular structure or sleevel 174. The outer structure 172 has a proximal end (not shown) and a distal end 172a. The inner sleeve 174 also has a proximal end (not shown) and a distal end 176. The inner sleeve is of slightly smaller diameter than the inner diameter of the tubular structure 172 to fit therein and be slidable therealong from a retracted position like that shown in FIG. 17a to an extended position like that shown in FIG. 17b. The extension of the inner sleeve from the retracted position to the extended position is accomplished by manipulating the two tubular structures 172 and 174 at their proximal ends. The distal end 176 of the inner sleeve 174 is in the form of a cutting tip, e.g., a sharp, beveled edge. This tip is arranged for piercing the cover material 22 of the covered stent 10 to provide an opening therein serving as an access port to a side branch otherwise blocked by the cover material.

Since the inner sleeve 174 of the piercing device is a hollow member, a conventional guidewire 64 can be extended though it, like shown in FIG. 17c. This enables one to provide guidewire access to a side-branch or bifurcated vessel which had been temporarily blocked by a portion of the cover of the covered stent, but which cover had been opened by the operation of the piercing tip 176.

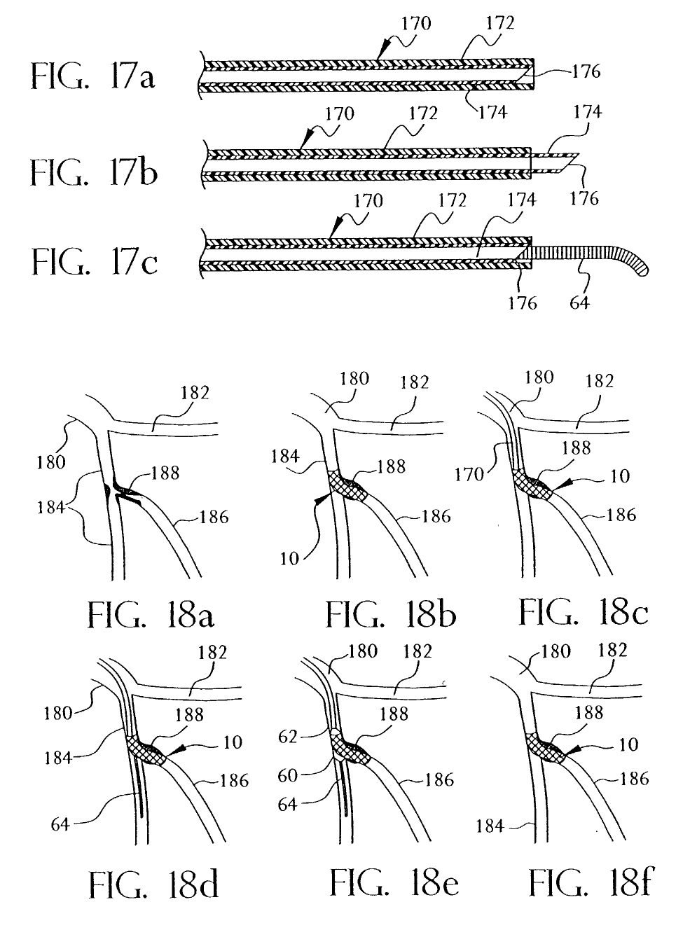

The use of the piercing device 170 is shown in FIGS. 18a-18f in a process of using a covered stent constructed in accordance with this invention to stent a lesion at the bifurcation of the left anterior descending (LAD) artery and the first diagonal branch (D1) off of the LAD to provide access to the LAD downstream of the bifurcation. To that end in FIG. 18a, there is shown the left main coronary artery 180, the circumflex branch 182, the left anterior descending artery (LAD) 184, and the first diagonal branch (D1) 186 off of the left anterior descending branch. There is a bifurcation lesion 188 in the LAD 184 and in D1 186. A covered stent 10 constructed in accordance with this invention is deployed in the LAD 184 and extends into D1 186 as shown in FIG. 18b. As discussed earlier since the cover 22 of the covered stent 10 is substantially solid, it will need to be modified in-vivo to open a passageway to the previously covered side-branch. Thus, when the covered stent 10 is deployed as shown in FIG. 18b, a portion of the cover will block the flow of blood into the LAD 184 downstream of the bifurcation. To open the covered stent so that blood can flow through the cover into the LAD, the piercing device 170 is used. In particular, as shown in FIG. 18c, the piercing device 170 is advanced to the portion of the covered stent 10 that is blocking the LAD 184. The inner sleeve 174 of the piercing device 170 is then operated to advance its cutting tip 176 through the cover material 20 between the struts 24 of the stent's framework. Once this has been accomplished a guidewire 64 can be extended through the hollow interior of the inner sleeve 174 such as shown in FIG.18d, whereupon the distal end of the guidewire passes through the covered stent and into the LAD 184 downstream of the stent. The guidewire 64 is left in place across the covered stent 10 while the piercing device 170 is removed. Next, a balloon catheter 62 is advanced into the LAD 184 over the guidewire 64 until the balloon 60 is partially across the covered stent 10, as is shown in FIG. 18e. The balloon 60 is then inflated to further open the opening through the cover 22 at the entrance to the LAD 184 optimize the flow into the distal or downstream portion of the LAD. In FIG. 18f, the covered stent 10 is shown in its fully deployed state to treat the lesion in the bifurcation and D1 186 and after the cover material 20 has been opened in the area of the LAD 184 to allow perfusion of the LAD.

Although the piercing device 170 shown is substantially straight, it is exemplary only. The piercing device may be angled or curved to facilitate entry into sidebranches or bifurcating vessels. Furthermore, the piercing device may be very flexible in construction to ensure a traumatic navigation through vessels.

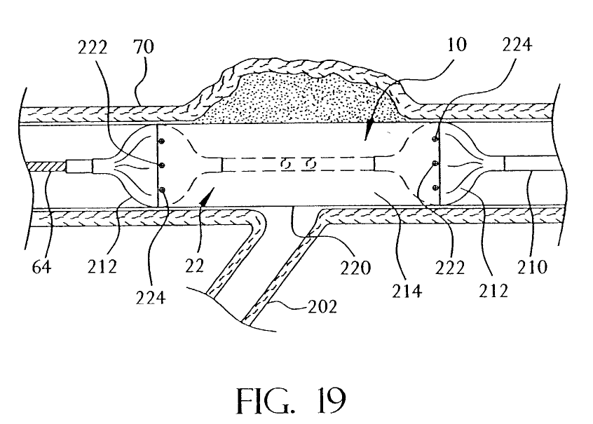

FIG. 19 shows a deployment system and methodology for selectively opening the cover of a covered stent, like than of FIG. 1 or constructed in accordance with any other embodiment of this invention and which is non-windowed, to provide access to a side-branch 202 temporarily blocked by the cover 22 of the stent. In particular, after the covered stent 10 is deployed in the target vessel 70, a double balloon catheter 210 can be placed over a guidewire 64 in the target vessel 70 across the occluded side-branch 202. The double balloons 212 on the catheter 210 can be inflated to isolate the area of the covered stent 10 which occludes the side-branch 202. The area between the balloons 214 is then infused with radiopaque fluid through infusion holes 216 to increase the pressure in the vessel 200 between the balloons. The increased pressure causes the cover material 22 to perforate or tear in the area 220 of the occluded side-branch 202. The radiopaque fluid can be seen in the side-branch vessel when the cover is perforated. If the ends of the covered double balloons are inflated against the stent, the pressure can be increased beyond the pressure which would exceed the elastic limit of the vessel, as long as the cover supported the vessel and kept the pressure on the vessel below that which would exceed its elastic limit. Matching radiopaque markers on the balloon catheter 222 and covered stent 224 could ensure proper alignment to ensure that the catheter was only increasing the pressure inside the covered stent. If the double balloon catheter was used outside the covered stent, the pressure in the vessel would have to be kept lower than the elastic limit of the vessel to ensure that vessel damage was not induced by the increased pressure.

As an alternative to or combined with the increased pressure between the balloons, heated fluid may be used to temporarily change the mechanical characteristics of the cover material to allow it to perforate or tear at a lower pressure. Heated fluid would be especially effective if the cover material is a thermoplastic. U.S Pat. No. 5,213,580 (Slepian) describes using heated fluid to render a resorbable polymer from a non-fluent state to a fluent state so mechanical force can be applied to the polymer to deform it into a desired shape.

Alternatively, only a single proximal balloon can be used to deliver heated or pressurized fluid into the vessel. Furthermore, if only heated fluid is necessary, a simple infusion catheter can be utilized to deliver heated fluid proximal to the covered stent.

It should be pointed out at this juncture that there are many possible methods of applying a cover to a stent structure as described previously. It has been demonstrated through experimentation that a very effective method of mounting a polymer cover to a metal stent structure involves using a very thin polymer film, preferably between 0.0005in and 0.002in, heating the polymer film above its glass transition temperature and wrapping the film around an unexpanded stent structure. This results in a very tightly wrapped film which is adequately adhered to the stent without the need for additional adhesive materials. Additionally, it is advantageous to wrap the cover around the stent after the stent has been crimped to a balloon. This prevents crimping of the stent after the cover has been mount to it which may result in separation of the cover from the stent.

As should be appreciated by those skilled in the art from the foregoing this invention provide a covered stent which is particularly suited for treating cardiovascular disease which overcomes the shortcomings of the prior art. Moreover, the covered stent can be used of other applications, as well. For example, the cover stents of this invention are particularly suited for mechanically support diseased or damaged lumens within the body of a living being. When used in a blood vessel they can increase blood flow in it. They can deliver drugs, other therapeutic agents, cellular material(s), genetic material(s) into a diseased or damaged lumen within a living being. They can be used to treat stenotic lesions which occur in the cardiovascular system. They can be used to seal aneurysms, perforations, and dissections in a blood vessel or other lumen. Moreover, the mechanical barrier provided by the stent cover can also prevent the release of debris through the stent struts which may cause distal embolization and the release of cyokines or other inflammatory agents into the vessel or lumen. They can be used to prevent distal embolization caused by release of debris from stent placement or post-stenting dilation, prevent the release of inflammatory agents from the vessel wall during percutaneous treatment. Further still the covered stents of this invention can be used to prevent restenosis of a vascular lesion, treat small diameter blood vessels, treat an acute myocardial infarction, a stroke, aortic aneurysms, degenerated saphenous vein grafts. All of this can be accomplished while preserving flow in side-branch vessels adjacent to or within the treatment area.

The stent structure may be a metal stent or polymer stent with a polymer cover. The stent may be formed by any conventional means including a coiled stent, slotted tube stent, self-expanding stent, or any other intravascular stent design. The polymer cover can be attached to the stent through a variety of means including wrapping a sheet of polymer material around the stent, or forming a tube of polymer material and mounting it over the stent. The polymer cover may be on the inside or outside of the stent or a combination thereof and may extend beyond the length of the stent. The stent cover may comprise one or more materials in its construction. The polymer material may be resorbable such a poly-lactic acid, poly-galatic acid or other resorbable polymers. The polymers may be formed by several different manufacturing processes, including extrusion, solvent casting, or compression molding. For deployment in the vasculature or other hollow organs, the stent with a cover may be balloon expandable or may be a self-expanding system. The stent cover is preferably very flexible as to not impact the flexibility of the stent or overall stent system and for applications where the stent is to expand is preferably very plastic and/or elastic so it can expand as the stent is deployed. The stent may have means of anchoring to the wall of the lumen it is deployed within.

The stent cover may incorporate drugs or other therapeutic or beneficial agents which would be released from the stent cover over a controlled period of time in-vivo. The drugs or agents may be anti-thrombogenic agents, anti-restenosis agents, anti-angiogenesis agents, anti-inflammatory agents or other agents. The stent cover may also incorporate cellular or other biological material to improve its therapeutic benefit. The incorporation of drugs or other biological material may be an additional means for preventing restenosis and thrombosis.

The stent cover may have properties to prevent permanent occlusion of a side-branch when placed within a branching vessel and may be constructed to selectively perforate or otherwise provide an opening to allow flow in a side-branch vessel.

Without further elaboration the foregoing will so fully illustrate our invention that others may, by applying current or future knowledge, adopt the same for use under various conditions of service.

We claim:

1. A covered stent for use in the body of a living being, said covered stent comprising a stent and a cover, said stent being an elongated tubular member having a pair of marginal edges and an intermediate portion and comprising plural elongated support portions interconnected with one another to form an open tubular framework, said open tubular framework having an inner surface and an outer surface, said cover being disposed over at least one of said surfaces of said hollow tubular framework and having an opening therein to enable said covered stent to be used in a vessel, duct, lumen or hollow organ of the living being that has a side-branch or bifurcation to another vessel, duct, lumen or hollow organ so that flow of a fluid from the vessel, duct, lumen or hollow organ to the other vessel, duct, lumen or hollow organ is not blocked by said cover.

2. The covered stent of claim 1 wherein said opening is disposed over plural elongated support portions of said framework.

3. The covered stent of claim 2 additionally comprising a radio-opaque marker located adjacent said opening.

4. A covered stent for use in the body of a living being, said covered stent comprising a stent and a cover, said stent being an elongated tubular member having a pair of marginal edges and an intermediate portion and comprising plural elongated support portions interconnected with one another to form an open tubular framework, said open tubular framework having an inner surface and an outer surface, said cover being disposed over at least one of said surfaces of said hollow tubular framework and having a penetratable portion to enable an opening to be formed in said penetratable portion to enable said covered stent to be used in a vessel, duct, lumen or hollow organ of the living being that has a side-branch or bifurcation to another vessel, duct, lumen or hollow organ so that flow of a fluid from the vessel, duct, lumen or hollow organ to the other vessel, duct, lumen or hollow organ is not blocked by said cover.

5. A covered stent for placement in a vessel, duct, lumen or hollow organ of a living being, said covered stent comprising a stent and a cover and at least two biologically active agents, said stent being an elongated tubular member having a pair of marginal edges and an intermediate portion and comprising plural elongated support portions interconnected with one another to form an open tubular framework, said open tubular framework having an inner surface and an outer surface, said cover being disposed over at least one of said surfaces of said hollow tubular framework, said cover comprising plural layers and having an outer surface, an inner surface and at least one intermediate surface, at least one of said biologically active agents being located on either or both of said exterior and interior surface, at least another of said at least two biologically active agents being located on said intermediate surface.

6. The covered stent of claim 5 wherein said plural layers of said cover are in the form of a sheet of material which has been rolled up to forms said plural layers.

7. The covered stent of claim 5 additionally comprising means to hold said plural layers together.

8. The covered stent of claim 5 wherein said cover is resorbable.

9. A system for deploying a stent within a vessel, duct, lumen or hollow organ in the body of a living being, the stent comprising a hollow expandable tubular member having a pair of marginal ends and an intermediate portion, said system comprising an inflatable balloon, said balloon being generally cylindrical in shape and having a distal end portion, and intermediate portion, and a proximal end portion, said balloon being arranged to support the stent thereon with the stent's end portion being located over said distal and proximal end portions of said balloon, and with the stent's intermediate portion being located over said intermediate portion of said balloon, said intermediate portion of said balloon being of greater diameter when inflated than said distal and proximal end portions of said balloon.

10. In combination an inflatable balloon and a stent, said stent comprising a hollow expandable tubular member having a pair of marginal ends and an intermediate portion, said balloon being generally cylindrical in shape and having a first portion and a second portion, said balloon being arranged to support said stent thereon with one portion of said stent being located over said first portion of said balloon, and with another portion of said stent being located over said second portion of said balloon, said first portion of said balloon being of greater diameter when inflated than said second portion of said balloon.
